(19) United States
(12) Patent Application Publication
(10) Pub. No.: US 2021/0137628 A1
(43) Pub. Date: May 13, 2021

(54) SYSTEMS FOR INDUCING AND INDICATING DEACTIVATION OF ADHESIVE DRAPES

(71) Applicant: KCI LICENSING, INC., San Antonio, TX (US)

(72) Inventors: Timothy Mark ROBINSON, Wimborne (GB); Christopher LOCKE, Bournemouth (GB); Ashwatha PRICE, Wimborne (GB)

(21) Appl. No.: 17/259,743

(22) PCT Filed: Jul. 18, 2019
(86) PCT No.: PCT/US2019/042317

§ 371 Date: Jan. 12, 2021

Related U.S. Application Data
(60) Provisional application No. 62/699,830, filed on Jul. 18, 2018.

(51) Int. Cl. A61B 46/20; A61B 46/00; A61L 31/06; A61L 31/14; A61L 31/04; C09J 7/30; C09J 9/00

(52) U.S. Cl. CPC A61B 46/20; A61B 46/40; A61L 31/06; A61L 31/14; A61L 31/048; C09J 7/30; C09J 2301/502; A61B 2046/205; C09J 2475/00; C09J 2433/00; C09J 2301/408; C09J 2301/416; C09J 9/00

(57) ABSTRACT

Provided herein are systems and methods for controlling, inducing, and indicating adhesive deactivation of adhesive drape systems. One aspect provides a system comprising a drape, a photosensitive adhesive layer, and a release agent, where the system is adapted to be coupled to a tissue site and released therefrom after exposure to an external stimulus, such as light. The system may have one or more photosensitive pigment areas that provide a visual indication based on exposure to the light. Another aspect provides a method for controlling the adhesion of a drape by application of a low-tack substance to one or more areas of a drape adhesive. The low-tack substance may be configured to produce a visual indication to distinguish areas where the low-tack substance has been applied. Such systems and methods enable a user to control properties of the drape adhesive and determine the various adhesive states via a visual indication.

### CROSS-REFERENCE TO RELATED APPLICATIONS

This application claims priority to and the benefit of U.S. Provisional Application No. 62/699,830, filed Jul. 18, 2018, the contents of which is incorporated into the present application by reference.

### BACKGROUND

### 1. Field of Invention

The present application relates generally to the field of tissue treatment, and more specifically to a system and method for facilitating the application and removal of a drape from a tissue site.

### 2. Description of Related Art

Systems and devices currently exist for the treatment of tissue, such as wound tissue and skin tissue. Some current tissue treatment systems require the use of an adhesive drape to secure all or a portion of the tissue treatment system to a tissue site. For example, an adhesive drape can be used to secure a gauze portion of a bandage to a wound site by adhering to the skin or other tissue surrounding the wound. Drapes intended for use with negative pressure wound therapy (NPWT) have certain desirable characteristics. Some of these characteristics are that the drape is easy to apply, doesn't adhere well to itself if folded (e.g., adhesive to adhesive) upon application to tissue, achieves a good seal with the tissue, adheres well to tissue and to its film (e.g., polyurethane) covering when layered or overlapped, enables atraumatic removal, is highly breathable, is repositionable upon application, and achieves adhesion that is not affected by patient heat or sweat.

### SUMMARY

Some dressings are designed and optimized for use on venous leg ulcers (VLUs). These types of wounds generally have very delicate peri-wound skin and can be damaged by exposure to strong adhesives and drapes. It is desirable to further optimize a dressing both for use under compression and not under compression. For delicate skin areas, it is a concern that acrylic adhesive may be too strong and that, when under compression, it may not be needed because the dressing would be held in place by the compression garment. On the other hand, for drapes not under compression, silicone dressings don't tend to adhere and seal adequately. Therefore, a VLU dressing or other drape technology using just silicone would not be able to maintain the seal necessary for NPWT. It is desirable to provide a drape that can enable the user to choose what adhesive works in what location at the time of application and be used both with and without compression.

Additionally, certain light sensitive or light deactivated adhesive drape systems have been proposed to allow easier removal of the drape system from a patient. Such adhesives have a strong bond strength to the skin when not exposed to certain wavelengths of light but deactivate upon exposure to the certain wavelengths of light. In some embodiments, the light deactivated adhesive can be deactivated upon exposure to certain wavelengths of visible light and, in other embodiments, can be deactivated upon exposure to certain wavelengths of non-visible light such as ultraviolet (UV) light. However, in these system, it is important that the user does not attempt to remove the drape from the wound until the adhesive has been fully deactivated. If the user attempts to remove the drape prematurely, the adhesive can damage the skin of the sensitive peri-wound area. Therefore, it is desirable to provide an indicator for use in light deactivated adhesive drape systems enabled to inform the user that the light sensitive adhesive is deactivated and the dressing is ready for removal. This can avoid any pain or discomfort caused due to premature removal of the dressing.

To alleviate the existing problems of the above disclosed systems, the disclosed embodiments describe a light deactivated adhesive drape system having a removable filter layer to protect the light deactivating adhesive drape system from premature deactivation upon inadvertent exposure to deactivation light wavelengths from ambient light. The illustrative embodiments described herein are directed to systems and methods for controlling, inducing, and indicating adhesive deactivation of certain adhesive drape systems. These embodiments can enable a user to fully optimize the adhesion of the dressing to the specific needs of the wound. Additionally, a range of differentiated bond-strength dressings can be provided that the user can selectively degrade for various applications.

Disclosed are various embodiments of a light deactivated adhesive drape system configured to be coupled to tissue. In some embodiments, the system comprises: a drape having an acrylic and/or polyurethane film, the film comprising: a photosensitive adhesive layer having at least one release agent disposed within the adhesive layer, wherein the at least one release agent is configured to weaken a bond of the adhesive layer to the tissue upon exposure to at least one of a plurality of light wavelengths; and a photosensitive pigment layer having at least one photo initiator disposed within the photosensitive pigment layer, wherein the at least one photo initiator is configured to change a color of the photosensitive pigment layer upon exposure to the at least one of the plurality of light wavelengths.

In some embodiments, the photosensitive pigment layer includes at least one of a dye and an ink. In some embodiments, the at least one photo initiator is configured to change from a first color that indicates that the bond of the adhesive layer is activated to a second color that indicates that the bond of the adhesive layer is deactivated. In some embodiments, the first color is blue and the second color is green. In some embodiments, the at least one photo initiator is configured to change to one or more intermediate colors after changing from the first color and before changing to the second color, wherein the one or more intermediate colors each indicate a deactivation state of the bond of the photosensitive adhesive layer. In some embodiments, the photosensitive pigment layer changes color at a same rate as a rate of deactivation of the bond of the photosensitive adhesive layer upon exposure to at least one of the plurality of light wavelengths. In some embodiments, the color changing rate is adjusted by changing a concentration of the photosensitive pigment layer. In some embodiments, the color changing rate is adjusted by adding one or more light screening agents to the photosensitive pigment layer. In some embodiments, the color changing rate is adjusted by covering the photosensitive pigment layer with at least one light attenuation layer. In some embodiments, the at least one light attenuation layer is a clear lacquer layer.

In some embodiments, the photosensitive adhesive layer is coated onto a surface of the acrylic and/or polyurethane film. In some embodiments, the photosensitive pigment layer is coated onto an opposite surface of the acrylic and/or polyurethane film. In some embodiments, the photosensitive pigment layer is disposed on at least one outer edge of the opposite surface of the acrylic and/or polyurethane film. In some embodiments, the photosensitive pigment layer comprises a plurality of dots including the at least one photo initiator. In some embodiments, the each of the plurality of dots includes a different concentration of the at least one photo initiator. In some embodiments, at least one light attenuation layer having a gradual thickness is disposed over the plurality of dots. In some embodiments, the photosensitive adhesive layer produces free radicals upon exposure to the at least one of the plurality of light wavelengths. In some embodiments, the photosensitive adhesive layer includes at least one free radical indicator that changes color upon exposure to the free radicals. In some embodiments, the photosensitive pigment layer includes at least one free radical indicator that changes color upon exposure to the free radicals. In some embodiments, the at least one photo initiator in the photosensitive pigment layer comprises one or more of chlorophyll, shikonin, and verdigris. In some embodiments, the plurality of light wavelengths includes wavelengths comprising blue through violet portions of the visible light spectrum. In some embodiments, the system further comprises a removable blocking layer configured to block the photosensitive adhesive layer from being exposed to the at least one of the plurality of light wavelengths. In some embodiments, the removable blocking layer comprises a single, opaque layer.

In some embodiments, a light deactivated adhesive drape system is configured to be coupled to tissue, the system comprising: an island dressing having an acrylic and/or polyurethane film, the film comprising: an absorbent portion configured to cover a wound on the tissue; a photosensitive adhesive layer having at least one release agent disposed within the adhesive layer, wherein the at least one release agent is configured to weaken a bond of the adhesive layer to the tissue upon exposure to at least one of a plurality of light wavelengths; and a photosensitive pigment layer having at least one photo initiator disposed within the photosensitive pigment layer, wherein the at least one photo initiator is configured to change a color of the photosensitive pigment layer upon exposure to the at least one of the plurality of light wavelengths. In some embodiments, the photosensitive pigment layer is coated onto an opposite surface of the acrylic and/or polyurethane film. In some embodiments, the photosensitive pigment layer is disposed on at least one outer edge of the opposite surface of the acrylic and/or polyurethane film.

In some embodiments, an adhesive drape system is configured to be coupled to tissue, the system comprising: a drape comprising: at least one acrylic and/or polyurethane adhesive portion configured to adhere to the tissue; and at least one selectably actuatable second adhesive portion configured to have an adhesion lower than an adhesion of the adhesive portion upon an actuation by a user, wherein the actuation decreases the adhesion of the adhesive portion to the tissue. In some embodiments, the actuation is an application of a low-tack compound to the at least one second adhesive portion. In some embodiments, the low-tack compound is directly applied to the drape. In some embodiments, the low-tack compound is directly applied to the tissue. In some embodiments, the low-tack compound is applied via a wipe. In some embodiments, the low-tack compound is a fluid silicone oil. In some embodiments, the low-tack compound is at least one of a suspension, emulsion, and a solution of at least one bio-compatible silicone polymer. In some embodiments, the at least one silicone polymer is at least one of a liquid, elastomer, thermoplastic, and a wax. In some embodiments, the at least one silicone polymer is at least one of a polyolefin, fluoropolymer, and fluorosilicone. In some embodiments, the low-tack compound includes at least one of a dye and a pigment. In some embodiments, the at least one of the dye and the pigment is at least one of methylene blue and indocyanine green.

In some embodiments, the at least one selectably actuatable second adhesive portion comprises a photosensitive adhesive that is deactivated upon exposure to at least one of the plurality of light wavelengths and wherein the actuation is an exposure of the at least one of the plurality of light wavelengths to the at least one second adhesive portion. In some embodiments, the exposure includes removing a removable blocking layer from the drape to expose the photosensitive adhesive to ambient light. In some embodiments, the exposure includes applying a light source to the photosensitive adhesive. In some embodiments, the photosensitive adhesive includes at least one of a photosensitive dye and a pigment. In some embodiments, the at least one of the photosensitive dye and the pigment is configured to change color to represent various adhesion states of the photosensitive adhesive, the adhesion states ranging from fully activated to fully deactivated. In some embodiments, the at least one of the photosensitive dye and the pigment is configured to change color at a same rate as a rate of deactivation of the photosensitive adhesive from a fully activated state to a fully deactivated state.

In some embodiments, the system further comprises a plurality of selectably actuatable second adhesive portions, wherein the plurality of selectably actuatable second adhesive portions have different grades of adhesion from each other. In some embodiments, the plurality of selectably actuatable second adhesive portions each include a different one of a plurality of a photosensitive dye and a pigment having a color corresponding to one of the different grades of adhesion. In some embodiments, one or more of the plurality of selectably actuatable second adhesive portions are actuated to provide a first grade of adhesion while a different one or more of the plurality of selectably actuatable second adhesive portions are actuated to provide a second grade of adhesion.

In some embodiments, a method comprises: coupling a drape system to a patient's tissue; actuating the at least one selectably actuatable second adhesive portion of the drape; and removing the drape from the tissue. In some embodiments, a method comprises: actuating at least one selectably actuatable second adhesive portion of the drape system; coupling the drape to a patient's tissue; and removing the drape from the tissue.

In some embodiments, a kit comprises: a drape system; and at least one wipe containing a low-tack compound. In some embodiments, the low-tack compound is a fluid silicone oil. In some embodiments, the low-tack compound is at least one of a suspension, emulsion, and a solution of at least one bio-compatible silicone polymer. In some embodiments, the at least one silicone polymer is at least one of a liquid, elastomer, thermoplastic, and a wax. In some embodiments, the at least one silicone polymer is at least one of a polyolefin, fluoropolymer, and fluorosilicone.

In some embodiments, a method comprises: coupling a light deactivated adhesive drape system to a patient's tissue; exposing the photosensitive adhesive layer to the at least one of the plurality of light wavelengths configured to weaken the bond of the adhesive layer; detecting that the photosensitive pigment layer has changed color to a color indicating that the drape is ready for removal; and removing the drape from the tissue. In some embodiments, the method further comprises removing the removable blocking layer from the drape system. In some embodiments, removing the removable blocking layer from the drape system comprises applying a solvent to the removable blocking layer and wiping away the removable blocking layer. In some embodiments, removing the removable blocking layer from the drape system comprises peeling off the removable blocking layer. In some embodiments, exposing the photosensitive adhesive layer to the at least one of the plurality of light wavelengths comprises exposing the photosensitive adhesive layer to at least one visible light wavelength. In some embodiments, the one visible light wavelength is in a blue through violet portion of the visible light spectrum. In some embodiments, the at least one photo initiator is configured to change to one or more intermediate colors after changing from a first color and before changing to a second color, wherein the one or more intermediate colors each indicate a deactivation state of the bond of the photosensitive adhesive layer. In some embodiments, the photosensitive pigment layer changes color at a same rate as a rate of deactivation of the bond of the photosensitive adhesive layer upon exposure to at least one of the plurality of light wavelengths.

The foregoing has outlined rather broadly the features and technical advantages of the present disclosure in order that the detailed description that follows may be better understood. Additional features and advantages of the disclosed embodiments will be described hereinafter that form the subject of the claims of the disclosure. It should be appreciated by those skilled in the art that the conception and specific embodiments disclosed may be readily utilized as a basis for modifying or designing other structures for carrying out the same purposes of the present disclosed embodiments. It should also be realized by those skilled in the art that such equivalent constructions do not depart from the spirit and scope of the disclosed embodiments as set forth in the appended claims. The novel features that are believed to be characteristic of the disclosed embodiments, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosed embodiments.

The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” and “about” may be substituted with “within [a percentage] of” what is specified, where the percentage includes 0.1, 1, 5, and 10 percent.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system, or a component of a system, that “comprises,” “has,” “includes” or “contains” one or more elements or features possesses those one or more elements or features, but is not limited to possessing only those elements or features. Likewise, a method that “comprises,” “has,” “includes” or “contains” one or more steps possesses those one or more steps, but is not limited to possessing only those one or more steps. Additionally, terms such as “first” and “second” are used only to differentiate structures or features, and not to limit the different structures or features to a particular order.

Any embodiment of any of the disclosed methods, systems, system components, or method steps can consist of or consist essentially of—rather than comprise/include/contain/have—any of the described elements, steps, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

### BRIEF DESCRIPTION OF THE DRAWINGS

Those of skill in the art will understand that the drawings, described below, are for illustrative purposes only. The drawings are not intended to limit the scope of the present teachings in any way.

FIGS. 1A-1B are cross-sectional views of a light deactivated adhesive drape system in accordance with an illustrative embodiment of the present disclosure.

FIGS. 2A-2C are cross-sectional views of a light deactivated adhesive drape system in accordance with another illustrative embodiment of the present disclosure.

FIGS. 3A-3C are cross-sectional views of an adhesive drape system in accordance with another illustrative embodiment of the present disclosure.

FIG. 4 is a flowchart illustrating a process for using a light deactivated adhesive drape system in accordance with an illustrative embodiment of the present disclosure.

FIG. 5 is a flowchart illustrating a process for using a light deactivated adhesive drape system in accordance with another illustrative embodiment of the present disclosure.

FIG. 6 is a flowchart illustrating a process for using an adhesive drape system in accordance with another illustrative embodiment of the present disclosure.

FIG. 7 is a flowchart illustrating a process for using an adhesive drape system in accordance with another illustrative embodiment of the present disclosure.

FIG. 8 is a top view of an adhesive drape system including an adhesive area and a pigment area in accordance with another illustrative embodiment of the present disclosure.

FIG. 9 is a top view of an adhesive drape system including an adhesive area and a pigment area in accordance with another illustrative embodiment of the present disclosure.

FIG. 10 is a top view of pigment areas of an adhesive drape system in accordance with another illustrative embodiment of the present disclosure.

FIG. 11 is a flowchart illustrating a process for using an adhesive drape system in accordance with another illustrative embodiment of the present disclosure.

### DETAILED DESCRIPTION

In the following detailed description of the preferred embodiments, reference is made to the accompanying drawings that form a part hereof, and in which is shown by way of illustration specific preferred embodiments in which the invention can be practiced. These embodiments are described in sufficient detail to enable those skilled in the art to practice the invention, and it is understood that other embodiments can be utilized and that logical structural, mechanical, electrical, and chemical changes can be made without departing from the spirit or scope of the invention. To avoid detail not necessary to enable those skilled in the art to practice the invention, the description can omit certain information known to those skilled in the art. It is understood that reference to a feature by numeric designation does not necessarily refer only to any particular embodiment depicted in a drawing. The following detailed description is, therefore, not to be taken in a limiting sense, and the scope of the present invention is defined only by the appended claims.

The following definitions are provided to better define the present invention and to guide those of ordinary skill in the art in the practice of the present invention. Unless otherwise noted, terms are to be understood according to conventional usage by those of ordinary skill in the relevant art.

Reduced pressure generally refers to a pressure less than the ambient pressure at a tissue site that is being subjected to treatment. In most cases, this reduced pressure will be less than the atmospheric pressure of the location at which the patient is located. Although the terms “vacuum” and “negative pressure” can be used to describe the pressure applied to the tissue site, the actual pressure applied to the tissue site can be significantly less than the pressure normally associated with a complete vacuum. Consistent with this nomenclature, an increase in reduced pressure or vacuum pressure refers to a relative reduction of absolute pressure, while a decrease in reduced pressure or vacuum pressure refers to a relative increase of absolute pressure.

As used herein, the term “coupled” includes “indirect coupling” via a separate object. For example, a drape can be coupled to the tissue site if both the drape and the tissue site are coupled to one or more third objects, such as a release agent or a second adhesive layer. The term “coupled” also includes “directly coupled,” in which case the two objects touch each other in some way. The term “coupled” also encompasses two or more components that are continuous with one another by virtue of each of the components being formed from the same piece of material. Also, the term “coupled” includes chemical coupling, such as via a chemical bond, and electrostatic coupling.

Various aspects of the present invention comprise systems and methods for controlling, inducing, and indicating adhesive deactivation of certain adhesive drape systems, a portion of which is shown in each of the FIGS. 1A-11. Various embodiments can facilitate the removal of the drape from the tissue site with less trauma to a patient than conventional drapes while preventing premature deactivation of the adhesive. The tissue site may be skin tissue, wound tissue, bone tissue, or any other type of tissue. Various embodiments of the system and method described herein comprise, or can be used with reduced or negative pressure wound healing technology.

Referring more specifically to FIGS. 1A and 1B, an illustrative embodiment of a light deactivated adhesive drape system 100 disposed onto patient tissue 104 is shown. The system 100 comprises a photosensitive adhesive layer 108 coupled to a flexible film layer 112. In some embodiments, the drape includes both adhesive layer 108 and flexible film layer 112. In the embodiments shown, a drape can be generally understood to be a covering over a tissue 104 that is preferably sterilizable. A drape can comprise a biocompatible thin film material, such as a polymer, a woven or non-woven material, an elastic or non-elastic material, an occlusive or nonocclusive material, and a flexible or inflexible material. A drape can comprise an impermeable, semi-permeable, or permeable material. Permeability characteristics can be selected according to desired moisture and gas (e.g., oxygen) transmission. In some embodiments, the drape comprises a material relatively impermeable to moisture and relatively permeable to oxygen. A drape can be coated with a material, for example, to control breathability. A drape can comprise a material which allows or facilitates transmission of external stimuli, such as light, sound, moisture or heat. For example, a drape material can be semi- or substantially transparent (i.e., pass or transmit more light that it blocks (e.g. reflects or absorbs)) to electromagnetic radiation, such as visible, ultraviolet (UV), or infrared light. A drape can be composed of one or more layers. In some embodiments, a drape can be a bilayer drape. For example, a bilayer drape can comprise flexible film layer 112 comprising any biocompatible thin film suitable for tissue or wound contact and a second layer 114 comprising a protective material. As another example, three, four, or more drape layers may be used, with combinations of materials selected according to desired function.

In the embodiment shown, the flexible film layer 112 may be a breathable and/or semiporous film such as polyurethane but other suitable materials may be used. The adhesive layer 108 adheres to the tissue 104 thereby coupling the flexible film layer 112 to the tissue 104. The adhesive layer 108 may cover any portion of the flexible film layer 112 and the tissue 104 as may be required. The adhesive layer 108 can comprise any material, in single or multiple layers, capable of adhering to tissue 104. In some embodiments, prior to the application of a drape to a tissue 104, the adhesive layer 108 can also be covered by an adhesive support layer (not shown). The adhesive support layer can provide rigidity to the drape prior to application and can also aid in the actual application of the drape onto tissue 104. The adhesive support layer can be peeled off or otherwise removed to expose adhesive layer 108 before applying the drape to the tissue. The adhesive layer 108 can comprise one or more materials including, but not limited to, polyurethane, acrylic (e.g., cyanoacrylate), hydrogel, silicon or silicone based material, natural rubber, synthetic rubber, styrene block copolymers, polyvinyl ethers, poly(meth)acrylates, polyolefins, hydrocolloid (e.g., a rubber based hydrocolloid), or a combination thereof. In some embodiments, the adhesive layer 108 comprises a polymer or co-polymer. For example, the adhesive layer 108 can comprise a co-polymer of polyurethane and silicone or various acrylic co-polymers.

The adhesive layer 108 may include at least one release agent 116 comprising a release material. In the embodiment shown, adhesive layer 108 has a plurality of release agents 116 (represented by dots in FIG. 1A). The release agent 116 can physically or chemically affect adhesion characteristics between a drape and a tissue 104. Adhesion characteristics may include tack, as measured by a tack test on stainless steel at room temperature, may include peel strength, as measured by a 180 degree angle peel test on stainless steel at room temperature, or both. A release agent 116 can comprise a variety of molecular compositions depending on the particular embodiment being implemented, including but not limited to a photopolymer, an oil particle, a gas particle, a solvent, a lipid, and/or one or more microstructures. Release agents 116 can be present in an inert or inactive form in, on, or near an adhesive layer 108. For example, a release agent 116 can be mixed with the adhesive; on the surface of the adhesive with a random or patterned coverage; coupled to the drape with a random or patterned coverage; or contained within a microstructure located in these or other locations. Upon release or activation, release agents 116 can migrate within the adhesive layer 108 or along an interface between an adhesive layer 108 and a tissue 104 to facilitate the removal of a drape affixed thereto. In the embodiment shown, the release agent 116 is configured to transition from an unreleased state (shown in FIG. 1A) to a release state 120 (represented by diagonal lines in FIG. 1B) to weaken a bond of the adhesive layer 108 to the tissue 104 upon exposure to an external stimulus. Various external stimulus can be employed depending on the particular embodiment being implemented. Non-limiting examples of the external stimulus include electromagnetic (e.g., UV, visible, or infrared light), magnetic, sound, pH, pressure (e.g., positive atmospheric pressure, negative atmospheric pressure, shear force, direct force), thermal, moisture, or a substance. The external stimulus can also be a substance, compound, liquid, or gas capable of reacting with a release agent 116 in adhesive layer 108 such that the release agent 116 transitions from an unreleased state to a released state. In the embodiment shown, the external stimulus is one or more of a plurality of light wavelengths. The weakened bond that occurs as a result of the release of release agent 116 allows a user of the light deactivated adhesive drape system 100 to apply an upward force on flexible film layer 112, such as a force indicated by arrow 124, to remove flexible film layer 112 from tissue 104. The weakened bond reduces the stress applied to tissue 104 in the removal of flexible film layer 112 from tissue 104. Thus, a patient feels less pain and discomfort when the flexible film layer 112 is removed. A residue of molecules from adhesive layer 108 might remain on tissue 104 after removal of flexible film layer 112 depending on a variety of factors such as the type of release agent used.

Referring more specifically to FIG. 1A, in the embodiment shown, release agents 116 are inertly dispersed within adhesive layer 108 and can be located anywhere within adhesive layer 108, as well as any of the outer surfaces of adhesive layer 108, such as an interface between adhesive layer 108 and flexible film layer 112. In some embodiments, release agents 116 can be bonded or coupled directly to flexible film layer 112, and a separate film layer (not shown in FIG. 1A), can separate release agents 116 from adhesive layer 108. In these embodiments, the presence of an external stimulus can weaken, break-down, or increase the permeability of the separate film layer such that release agents 116 are allowed to migrate into adhesive layer 108 to facilitate the removal of flexible film layer 112 from tissue site 105. As shown in FIG. 1B, release agents 116 may be released in the presence of external stimulus such that release agents 116 are allowed to migrate within adhesive layer 108 and the interface between adhesive layer 108 and tissue 104. In the embodiment shown, a UV light source 128 exposes flexible film layer 112 and adhesive 108 to a plurality of light wavelengths 132. In some embodiments, exposure to the plurality of light wavelengths 132 can cause microstructures containing release agents 116 to rupture or tear, thereby releasing release agents 116 from the interior of the microstructures. These released release agents 116 can then be interspersed into adhesive layer 108 and the interface between adhesive layer 108 and tissue 104, thereby weakening the bond between flexible film layer 112 and tissue 104 and facilitating the removal of flexible film layer 112 from tissue 104. As the plurality of light wavelengths 132 reach adhesive 108, release agents 116 may transition from an unreleased state (as shown in FIG. 1A) to a released state 120 (as shown in FIG. 1B) as they are exposed to the plurality of light wavelengths 132. In the embodiment shown, the plurality of light wavelengths 132 are UV wavelengths. In some embodiments, the UV wavelengths may be within a range of 280 nm-380 nm, although it may be preferable to have the UV wavelengths be UVA wavelengths within a range of 315 nm-380 nm.

Referring now to FIGS. 2A-2C, another illustrative embodiment of a light deactivated adhesive drape system 200 disposed onto patient tissue 104 is shown. In this embodiment, light deactivated adhesive drape system 200 is configured to release adhesive layer 108 upon exposure to ambient, visible light instead of UV light. Although having adhesive layer 108 release upon exposure to visible light is advantageous in that it doesn't require a specific UV light source and enables release to occur in any environment having ambient light, it also can increase a likelihood that the adhesive layer 108 will prematurely deactivate. In order to prevent premature deactivation, the adhesive layer 108 may be constructed with release agents 116 that only release upon exposure to certain wavelengths of visible light. For example, in the embodiment shown, release agents 116 will only transition to an unreleased state 120 when exposed to visible light wavelengths in the blue through violet portions of the visible light spectrum. In the embodiment shown, a blocking layer 204 is disposed over flexible film layer 112 of the drape. In this embodiment, blocking layer 204 is configured to block the light wavelengths that release the adhesive layer 108.

In the embodiment shown in FIG. 2B, filter layer 204 is configured to be removable (e.g., by providing a force represented by arrows 208, by peeling, and by other suitable methods of removal). This enables the adhesive layer 108 to be deactivated at a time a user desires to remove the drape from tissue 104. As shown in FIG. 2C, once the filter layer 204 has been removed, the adhesive layer 108 can be exposed to deactivation wavelengths (e.g., ambient or UV light 216) that comprises light wavelengths configured to deactivate adhesive layer 108. Upon exposure to light 216, release agents 116 can transition from an unreleased state to a released state 120. The drape including flexible film layer 112 can then be removed from tissue 104 (e.g., by providing a force represented by arrows 124). If any residue of adhesive layer 108 remains on tissue 104 after removal of the drape, it may be removed.

Referring now to FIGS. 3A-3C, another illustrative embodiment of an adhesive drape system 300 disposed onto patient tissue 104 is shown. System 300 is configured to enable a user to actively and selectably control the adhesion of the drape system 300 to the patient tissue 104. In the embodiment shown, an absorbent portion or region 304 is placed in contact with the skin tissue, usually covering the wound site. The absorbent portion 304 can be provided as a separate element from the rest of the dressing as shown or may be provided as part of a unitary wound covering along with the adhesive regions 308, 312. In the embodiments shown, adhesive regions 308, 312 comprise regions having different adhesive tack strength from each other. This can be any combination of acrylic adhesive, light deactivated and/or photosensitive adhesive, and/or low adhesive polymers or other compounds. For example, in the embodiment shown in FIG. 3A, adhesive regions 308 can be portions constituting a low adhesive or tack substance. These low adhesive regions can be disposed at various areas or intervals along an underside of flexible film layer 112 and intermittently with adhesive regions 312. In the embodiment shown, adhesive regions 312 have a higher adhesion (e.g., tack, peel strength, or both) than adhesive regions 308.

In the embodiment shown in FIG. 3A, the user can control/selectively actuate the different levels of adhesion of adhesive regions 308, 312 by manually using a wipe 316 or other delivery method of a suitable low-tack compound to either block or otherwise render non-functional the higher tack adhesive in specific locations. Low as used in low-tack compound refers to the tack level of the compound being less than a tack level of the adhesive that forms adhesive portions 312 (e.g., acrylic adhesive, such as high-tack acrylic adhesive). Alternatively, a low-tack compound may be a chemical that lowers or alters a tack level of the adhesive that forms adhesive portions 312 (e.g., acrylic adhesive, such as high-tack acrylic adhesive) itself, such as by chemical interaction. High-tack acrylic adhesive corresponds to adhesive that has a tack level (and optionally corresponding peel strength) that may cause discomfort to the skin during removal if the high-tack acrylic adhesive is moved shortly after application at full tack (e.g., before degradation of tack and/or bond strength), is not used in combination with another adhesive (e.g., the adhesive of adhesive regions 308) or is not weakened prior to removal.

A low-tack compound may thus cause a lower tack by chemical or physical interaction with the adhesive or the low-tack compound may provide an intermediate layer to which the adhesive, the skin, or both bond. As the layers or materials with the lowest bond will separate first, the higher tack of the adhesive portions 312 will not be directly bound to the tissue or a reduced portion of the adhesive portions 312 be bound to the tissue, effectively reducing the tack and peel strength of the adhesive even though its tack, bond strength, or theoretical peel strength has not been altered.

In some embodiments, the wipe or other delivery device can be included in a kit that also contains the adhesive drape system. For example, adhesive areas 308 may initially comprise the same higher tack adhesive that forms adhesive portions 312 (e.g., high-tack acrylic adhesive). If the user desires to modify or deactivate certain areas of the high-tack adhesive portions 312 in order to reduce the total adhesion of the drape to the patient tissue 104, the user can wipe one or more areas of adhesive portions 312 with wipe 316 to change them into low-tack adhesive portions 308. Alternatively or additionally, the user can wipe one or more areas of the patient tissue surrounding absorbent portion 304 with wipe 316 to deposit a low-tack compound or substance onto the skin. The low-tack compound can change certain areas of adhesive portions 312 to low-tack adhesive portions 308 upon contact with those areas, thus enabling the user to selectably actuate the low-tack adhesive portions 308. In this way, the low-tack compound may be applied to peri-wound areas of the patient tissue 304 directly or may be pre-applied to certain adhesive portions (e.g., 308) of the dressing before placement of the system 300 onto the patient tissue 104.

In some embodiments, the low-tack compound may be one or more of a fluid silicone oil, or a suspension, emulsion, or solution of a silicone polymer with inherently low adhesive tack. Other low-tack polymers may be used such as polyolefins, fluoropolymers, and fluorosilicones. The polymers may be liquids, elastomers, thermoplastics, or waxes. The polymers can also be fully bio-compatible with patient tissue 104. Mineral dispersions, such as particulate carbonates (e.g., calcium and magnesium), clays, and silicates may be used instead of or in combination with the polymers listed above. Long chain surfactants in solution may also be used. Aerosol dispensers may be used to apply the low-tack compound instead of wipe 316, particularly if a large area needs to be covered.

Once the user has applied the low-tack compound to one or more of the skin and/or the dressing, the user can adhere the drape to the patient tissue 104 by applying, for example, a force in the direction indicated by arrows 320. This configuration is shown in FIG. 3B. Flexible film layer 112 covers and holds or maintains absorbent portion 304 against the wound while adhesive portions 308, 312, adhere the flexible film layer 112 in place on patient tissue 104. In the embodiment shown, higher tack adhesive portions 312 provide all or most (e.g., over 50%) of the total adhesion force between flexible film layer 112 and patient tissue 104. Lower tack adhesive portions 308 reduce the total adhesion between flexible film layer 112 and patient tissue 104 in order to decrease possible trauma to sensitive peri-wound areas upon desired removal of the drape.

In some embodiments, different adhesion grades of compound may be used to selectively degrade the adhesive function in addition to selectively switching or deactivating it completely. For example, adhesive portions 308 can comprise a diluted adhesive compound that degrades the adhesion strength of adhesive potions 312 to 50% or any other % deemed appropriate. Referring to FIG. 3B, a user may choose to deactivate the adhesive completely in one location (e.g., right side adhesive portion 308), degrade the adhesive in another location (e.g., left side adhesive portion 324), and leave a third region with 100% original adhesive bond (e.g., left side adhesive portion 312). In this way, the user can tailor the total adhesion of the drape for application to various tissue areas and for various application times.

In some embodiments, one or more light deactivated adhesives may be used as the adhesive for adhesive portions 308 and/or 312. For example, referring to the embodiment shown in FIG. 3C, high-tack adhesive regions 312 can be light deactivated adhesive portions having high tack when activated and low tack when deactivated. A user can apply deactivating light 212 to the light deactivated adhesive portions when removal of the drape is desired to convert the light deactivated adhesive portions to a deactivated state 328. For example, the user can use a UV light source to expose the drape to UVA and the light deactivated adhesive portions would gradually lose bond strength. Alternatively, the user can remove one or more blocking layers to expose the drape to ambient visible light to deactivate the light deactivated adhesive portions. In some embodiments, different types of light deactivated adhesive portions can be used that are sensitive to different wavelengths (e.g., one portion may be deactivated with UV light and another portion may be deactivated with visible light in the red and/or orange part of the visible light spectrum). Upon deactivation of the light deactivated adhesive portions, the user can remove the drape and all or most of the adhesive portions 308, 312 (e.g., by providing a force represented by arrows 124).

Different combinations of adhesive types can be used depending on the application and/or type of the drape. For example, adhesive portions 308 can be formed of one type of light deactivated adhesive while adhesive portions 312 are formed of another type of light deactivated adhesive. In these embodiments, adhesive portions 308 may deactivate at a different rate than adhesive portions 312. In other embodiments, adhesive portions 308 can be formed of a light deactivated adhesive while adhesive portions 312 are formed of a high-tack acrylic adhesive.

FIG. 4 depicts a flowchart illustrating a general process 400 for facilitating removal of a light deactivated adhesive drape system from a tissue 104 in accordance with an illustrative embodiment of the disclosure. The process illustrated in FIG. 4 can be implemented by a user of a reduced or negative pressure treatment system. The process begins by applying a drape to a tissue 104 (step 404). In this step, adhesive layer 108 can bind to the tissue 104. Also in this step, reduced or negative pressure can be applied to the tissue 104 using a reduced or negative pressure treatment system. The process determines whether to remove the drape from the tissue 104 (step 408). If the process determines not to remove the drape from the tissue 104, the process returns to step 408. If the process determines to remove the drape from the tissue 104, the process applies an external stimulus to the drape, including the adhesive layer 108 coupled to the drape (step 412). In this step, a release agent 116 can be released in accordance with any of the illustrative embodiments described above to facilitate the removal of the drape from the tissue 104. The process then removes the drape from the tissue 104 (step 416).

FIG. 5 depicts a flowchart illustrating a process 500 for facilitating removal of a light deactivated adhesive drape system (e.g., 100, 200, or 300) from a tissue 104 in accordance with an illustrative embodiment of the disclosure. Referring to FIG. 5, process 500 begins by coupling a drape to a tissue (step 504). In this embodiment, the drape may have a removable blocking layer that is configured to be removed at a desired removal time of the drape. Process 500 continues by, when the drape is desired to be removed, the removable blocking layer is removed from the drape (step 508). Process 500 continues by applying certain deactivating light wavelengths to the photosensitive adhesive layer to deactivate the adhesive (step 512). The process then enables a removal of the drape from the tissue (step 516).

FIGS. 6 and 7 depict flowcharts illustrating a processes 600, 700 for facilitating removal of a user controllable drape system (e.g., 300, including drapes with light deactivated adhesive portions) from a tissue 104 in accordance with an illustrative embodiment of the disclosure. Referring to FIG. 6, process 600 begins by applying a low-tack adhesive compound to the drape and/or to the patient tissue (step 604). Process 600 continues by coupling the drape to a tissue (step 608). The process 600 then enables a removal of the drape from the tissue (step 612), such as after use of the drape. Referring to FIG. 7, process 700 begins by coupling a drape to a tissue (step 704). Process 700 continues by deactivating certain adhesive portions of the drape (step 708) , such as after use of the drape. In some embodiments, these certain adhesive portions can be light deactivated adhesive portions that are deactivated by applying certain deactivating light wavelengths to the light deactivated adhesive portions. The process 700 then enables a removal of the drape from the tissue (step 712).

FIGS. 8 and 9 depict drapes 800 and, 900, drapes 800 and 900 each have one or more adhesive areas (804, 904) and one or more photosensitive pigment areas (808, 908). In some embodiments, one or more photosensitive pigment areas (808, 908) are included as part of a photosensitive pigment layer. In the embodiments shown, a dye/ink/pigment may be used in the dressing along with the various adhesives to indicate an adhesive state to the user. In embodiments using an acrylic adhesive, particles of pigment can be disposed into the adhesive. These pigments are configured to change color as the adhesion state of the adhesive changes or is modified. For example, an adhesive portion having a 100% adhesion state (e.g., a fully activated state) or a high-tack adhesion state (e.g., a fully activated state) may be a green color and an adhesive portion having a 50% adhesion state or a lower tack adhesion state (e.g., a fully deactivated state) may be a blue color. This change in color can be triggered by application of a low-tack compound by the user.

Using the embodiments shown in FIG. 3A and FIG. 8 as examples, if a user applies a low-tack compound to portions of the adhesive (e.g., adhesive portions 308), the pigment in those portions will change color to indicate to the user that those portions are in a low-tack adhesion state (e.g., pigment areas 808). The pigment in the portions where a high-tack adhesion state remains (e.g., adhesive portions 312) would remain in the original color (e.g., adhesive area 804). In some embodiments, the low-tack compounds (instead of the adhesive) can have the colored dyes/pigments disposed in them so that the user can distinguish the areas where the low-tack compound has been applied (e.g., adhesive portions 308) from the areas where it has not been applied (e.g., adhesive portions 312).

In embodiments where the user desires selective adhesive degradation, each different low-tack compound can have a different colored pigment to represent each grade of the selective degradation and the areas where each low-tack compound have been applied. In some embodiments, the dye/pigment can be a visible or wavelength luminous dye/pigment that can be used to indicate where the low-tack compound has been applied, and can comprise materials such as methylene blue and indocyanine green.

In embodiments using a light deactivated switchable adhesive, a photosensitive dye/ink/pigment having at least one photo initiator may be used to indicate the adhesive state to the user. In these cases, the light deactivated switchable adhesive, when it is exposed to and reacts to the visible light to deactivate the adhesive, will change color, for example, from blue to green. For example, when the dressing is placed on the wound, the original color of the adhesive is blue and, when exposed to light, it turns green after the desired exposure time. This change of color indicates to the user that the adhesive has been deactivated and the dressing is ready for removal. Exemplary types of photosensitive pigment that can be used are chlorophyll, shikonin, or verdigris

In FIG. 8, a schematic top view illustrating adhesive and photosensitive material placement is shown. In the example embodiment shown in FIG. 8, a light sensitive adhesive may be coated onto a first side 812 of a flexible film layer (e.g., polyurethane film) to form adhesive area 804 and photosensitive dye/ink/pigment may be coated onto a second side 816 (e.g., the opposite side) of flexible film layer. As illustrated in FIG. 8, the photosensitive dye/ink/pigment is coated on one or more outer edges of the second side 816 of the flexible film layer to form pigment areas 808. In the embodiment shown, the adhesive area 804 is disposed on the first side 812 which is the tissue facing side of the drape and the pigment areas 808 are disposed on the second side 816 which is the opposite side facing away from the tissue. In this embodiment, the light sensitive adhesive coated flexible film bonds to the skin and the areas 808 of the flexible film having the photosensitive dye/ink/pigment are not in contact (e.g., not in direct contact) with the skin. In some embodiments, the flexible film layer can be covered to be lightly bonded to a breathable/flexible, opaque, and/or removable light blocking layer. Opaque may correspond to a material or layer that reflects or absorbs a substantial or majority portion of a particular type of light and/or that does not transmit a substantial or majority portion of the particular type of light.

When using the embodiment shown in FIG. 8, the user can remove one or more release layers (e.g., cover films) and apply the dressing to patient tissue. When the user wishes to remove the dressing, the user can remove the removable blocking layer to expose the light sensitive adhesive areas 804 and the photosensitive pigment areas 808 to the deactivating light wavelengths. As the light sensitive adhesive areas 804 move from a high-tack adhesion state to a low-tack adhesion state as the light sensitive adhesive is deactivated, the photosensitive pigment areas 808 will change from a color indicating a high adhesion state (i.e., activation state) to a color indicating a low adhesion state (i.e., deactivation state). After a prescribed length of time (depending on the light intensity and the type/concentration of the adhesive used), photosensitive pigment areas 808 will change color to indicate to the user that the light sensitive adhesive areas 804 have been deactivated and the dressing is able to be removed from the tissue without trauma.

In some embodiments, the photosensitive pigment areas 808 have two colors, one representing an adhesive activation or high-tack state and the other representing an adhesive deactivation or low-tack state. In this embodiment, the photosensitive pigment areas 808 will not change to the color representing the adhesive deactivation or low-tack state until the adhesive areas 804 are fully deactivated and the dressing is ready for removal. In other embodiments, the photosensitive dye/ink/pigment may be designed to react to the visible light at a similar rate as the light sensitive adhesive. Certain types of photosensitive dyes (e.g., Reversacol Palatinate Purple™ and Reversacol Plum™ available from Vivimed Labs of Hyderabad, India) can be used to react to visible light at the same rate or a similar rate as certain light deactivated adhesives. The rate of color change may be adjusted or “tuned” to that of the adhesive reaction to light by methods such as changing the concentration of the light sensitive dye, mixing the dyes with light screening agents (such as titanium dioxide), or covering the dye print with additional clear lacquer light attenuation layers (to attenuate the light dose). A clear lacquer light attenuation may include or correspond to layer which attenuates light but still enables a person to visual see through the layer. For example, the layer may be transparent or translucent to visible light (or a portion thereof) and opaque to UV light (i.e., reflect or absorb a substantial or majority portion of UV light and/or not transmit a substantial or majority portion of UV light). The adhesive deactivation can be shown by a color change in which the color hue would increase in depth as the exposure increased. In these embodiments, the user would stop exposure when the level of bond strength was achieved (e.g., as judged by the color displayed and confirmed by reference to a chart).

Referring to the embodiment shown in FIG. 9, drape 900 can be an island dressing having an adhesive area 904, one or more pigment areas 908, and an absorbent area/portion 912. In the embodiment shown, adhesive area 904 can be disposed continuously to surround a border of absorbent area 912 to form an “island” and the pigment areas 908 can be disposed at an outer edge of the drape to at least partially surround the central absorbent area/portion 912. Similar to the embodiment of FIG. 8, adhesive area 904 can be an acrylic adhesive and pigment areas 908 can represent areas where a low-tack adhesive or low-tack compound has been applied by a user. Adhesive area 904 can also be a light sensitive adhesive that deactivates upon exposure to certain light wavelengths and pigment areas 908 can comprise a photosensitive dye/ink/pigment that changes color upon exposure to the certain light wavelengths.

In some embodiments, photo initiators in the light sensitive adhesive produce free radicals when exposed to deactivating light wavelengths. In these embodiments, a mechanism may be used where a photosensitive dye/ink/pigment reacts with these free radicals to change colors. Free radical or redox indicators such as methylene blue and/or neutral red may be used. The redox indicators can be mixed with the light sensitive adhesive before coating. In some embodiments, the light sensitive adhesive can be applied to a patient tissue facing side of the flexible film layer and the photosensitive dye/ink/pigment configured to react to free radicals may be applied to the opposite side of the flexible film layer that faces away from the patient tissue. This configuration has the advantage of not necessarily reducing the activity of the light sensitive adhesive (in contact with the skin) due to having a portion of its generated free radicals reacting with the light sensitive dye. The dye “doped” adhesive would thus not require “tuning” as discussed above to get it to react at the same rate as the light sensitive adhesive since both the adhesive and pigment use the same functional materials. This dye “doped” adhesive may also be used to permit bonding of the blocking layer, and could be applied in a pattern to control the required bond strength.

As shown in the embodiment of FIG. 10, a drape 1000 having a dosimeter design with a flexible film layer 1004 having multiple dots 1008, referred to generally as dots 1008 including first through third dots 1012, 1016 ,1020, of photosensitive dye/ink/pigment disposed onto or within different areas of the flexible film layer 1004. The multiple dots 1008 can be used to indicate various levels of deactivation of the light sensitive adhesive. This design can display to the user that an adhesive deactivation process has commenced and inform the user of the deactivation state various areas of the adhesive have reached. This design can also be used with different pigments to show areas where a low-tack compound has been applied by a user to an acrylic adhesive.

In the embodiment shown, dots 1008 disposed on the different areas of flexible film layer 1004 are each different colors and/or different hues of the same or different color. For example, first dot 1012 has a light hue and represents a high adhesive state of the drape. This light hue can indicate that a low-tack compound has not been or only lightly applied to an adhesive area or that a light sensitive adhesive has not been or only slightly deactivated. Second dot 1016 has a medium hue and represents an intermediate adhesive state of the drape. This medium hue can indicate that an intermediate amount or concentration of low-tack compound has been applied to an adhesive area or that a light sensitive adhesive is in the process of being deactivated but has not yet reached a fully deactivated state. Third dot 1020 has a dark hue and represents a low adhesive state of the drape. This dark hue can indicate that a high amount or concentration of low-tack compound has been applied to an adhesive area or that a light sensitive adhesive has reached a fully deactivated state that can enable a removal of the drape without patient trauma.

Dots 1008 can represent different concentrations of photosensitive pigments and/or different types of pigments. Dots 1008 can also represent areas of drape 1000 where different amounts or concentrations of low-tack compounds have been applied, different areas/layers of a film having graduated thicknesses to attenuate the light levels reaching photosensitive pigments (i.e., light attenuation layers), or different levels or types of adhesive achieved through printing techniques. The embodiment shown in FIG. 10 may be especially useful to represent different adhesion states when a selective gradation of adhesive levels is desired by the user. The drapes 800, 900, and 1000 may be included or incorporated into drape systems 100, 200, or 300. Additionally, or alternatively, features of the drapes and 800, 900, and 1000 and drape systems 100, 200, or 300 may be mixed and matched to generate embodiments consistent with the exemplary example embodiments illustrated in the above described figures.

FIG. 11 depicts a flowchart illustrating a process 1100 for facilitating removal of a user controllable drape system (e.g., a drape system, such as 100, 200, or 300, including a drape, such as 800, 900, or 1000) using photosensitive pigments from a tissue 104 in accordance with an illustrative embodiment of the disclosure. Referring to FIG. 11, process 1100 begins by coupling the drape to a tissue (step 1104). Process 1100 continues by applying a deactivating medium to certain adhesive portions of the drape (step 1108). In some embodiments, these certain adhesive portions can be light deactivated adhesive portions that are deactivated by applying certain deactivating light wavelengths to the light deactivated adhesive portions. In other embodiments, the deactivating medium can be a low-tack compound applied by a user to certain adhesive portions of the drape. Process 1100 continues by detecting a color and/or hue of a pigment layer/area (step 1112). In some embodiments, the pigment area comprises a photosensitive pigment that changes color to correspond to a deactivation state of a light sensitive adhesive. In other embodiments, a certain color of a pigment area designates portions where a low-tack compound has been applied. The process 1100 then enables a removal of the drape from the tissue (step 1116). Removal of the drape can be performed at a time when photosensitive pigment indicates that a light sensitive adhesive has been fully deactivated or when a certain pigment color indicates that enough low-tack compound has been applied to the adhesive layer of the drape to enable low trauma removal of the drape from the patient tissue.

The flowcharts and block diagrams in the different depicted embodiments illustrate the architecture, functionality, and operation of some possible implementations of the apparatus and methods. Such flowcharts and block diagrams include FIGS. 4-7 and 11. Such flowcharts and block diagrams may be useable with the drapes of FIGS. 1A, 1B, 2A-2C, 3A-3C, and 8-10. In some alternative implementations, the function or functions noted in the block can occur out of the order noted in the figures. For example, in some cases, two blocks shown in succession can be executed substantially concurrently, or the blocks can sometimes be executed in the reverse order, depending upon the functionality involved. Additionally or alternatively, in some other implementations, a function of one or more blocks may be omitted. Similarly, a function of a block of another method or device described herein can be added.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the disclosed methods, devices, and systems are not intended to be limited to the particular forms disclosed. Rather, they include all modifications and alternatives falling within the scope of the claims, and embodiments other than those shown may include some or all of the features of the depicted embodiment. For example, components may be combined as a unitary structure and/or connections may be substituted. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A light deactivated adhesive drape system configured to be coupled to tissue, the system comprising:
a drape having an acrylic and/or polyurethane film, the film comprising:
a photosensitive adhesive layer having at least one release agent, wherein the at least one release agent is configured to weaken a bond of the adhesive layer to the tissue upon exposure to at least one of a plurality of light wavelengths; and
a photosensitive pigment layer having at least one photo initiator, wherein the at least one photo initiator is configured to change a color of the photosensitive pigment layer upon exposure to the at least one of the plurality of light wavelengths.

2. The system of claim 1, wherein the photosensitive pigment layer includes at least one of a dye and an ink, and wherein the at least one release agent, the at least one photo initiator, or both are disposed within the photosensitive pigment layer.

3. The system of claim 1, wherein the at least one photo initiator is configured to change from a first color that indicates that the bond of the adhesive layer is activated to a second color that indicates that the bond of the adhesive layer is deactivated.

4. The system of claim 3, wherein the first color is blue and the second color is green.

5. (canceled)

6. The system of claim 1, wherein the photosensitive pigment layer is configured to change color at a same rate as a rate of deactivation of the bond of the photosensitive adhesive layer upon exposure to at least one of the plurality of light wavelengths.

7-8. (canceled)

9. The system of claim 6, wherein the color changing rate is based on at least one light attenuation layer coupled to the photosensitive pigment layer.

10. The system of claim 9, wherein the at least one light attenuation layer is a clear lacquer layer.

11. The system of claim 1, wherein the photosensitive adhesive layer is positioned on a first surface of the acrylic and/or polyurethane film.

12. The system of claim 11, wherein the photosensitive pigment layer is positioned on a second surface opposite the first surface of the acrylic and/or polyurethane film.

13. The system of claim 12, wherein the photosensitive pigment layer is positioned on at least one outer edge of the second surface of the acrylic and/or polyurethane film.

14. The system of claim 13, wherein the at least one photo initiator of the photosensitive pigment layer is arranged into a plurality of dots.

15. The system of claim 14, wherein at least one dot of the plurality of dots includes a different concentration of the at least one photo initiator from at least one other dot of the plurality of dots.

16. The system of claim 14, wherein at least one light attenuation layer has a varying thickness and is position over the plurality of dots.

17. The system of claim 1, wherein the photosensitive adhesive layer is configured to produce free radicals upon exposure to the at least one of the plurality of light wavelengths.

18. The system of claim 17, wherein the photosensitive adhesive layer includes at least one free radical indicator configured to change color upon exposure to the free radicals.

19. The system of claim 17, wherein the photosensitive pigment layer includes at least one free radical indicator configured to change color upon exposure to the free radicals.

20-23. (canceled)

24. A light deactivated adhesive drape system configured to be coupled to tissue, the system comprising:
an island dressing having an acrylic and/or polyurethane film, the film comprising:
an absorbent portion configured to cover a wound on the tissue;
a photosensitive adhesive layer having at least one release, wherein the at least one release agent is configured to weaken a bond of the adhesive layer to the tissue upon exposure to at least one of a plurality of light wavelengths; and
a photosensitive pigment layer having at least one photo initiator,
wherein the
at least one photo initiator is configured to change a color of the photosensitive pigment layer upon exposure to the at least one of the plurality of light wavelengths.

25. The system of claim 24, wherein the photosensitive pigment layer is coated onto an opposite surface of the acrylic and/or polyurethane film.

26. The system of claim 25, wherein the photosensitive pigment layer is disposed on at least one outer edge of the opposite surface of the acrylic and/or polyurethane film.

27. An adhesive drape system configured to be coupled to tissue, the system comprising:
a drape comprising:
at least one acrylic and/or polyurethane adhesive portion configured to adhere to the tissue; and
at least one selectably actuatable second adhesive portion configured to have an adhesion lower than an adhesion of the adhesive portion upon an actuation by a user,
wherein the actuation decreases the adhesion of the adhesive portion to the tissue.

28. The system of claim 27, wherein the at least one selectably actuatable second adhesive is configured to be actuated by a low-tack compound.

29. The system of claim 28, further comprising a wipe comprising the low-tack compound.

30. The system of claim 29, wherein the wipe is configured to directly apply the low-tack compound to the drape.

31. The system of claim 29, wherein the wipe is configured to directly apply the low-tack compound to the tissue.

32-37. (Canceled)

38. The system of claim 27, wherein the at least one selectably actuatable second adhesive portion comprises a photosensitive adhesive that is deactivated upon exposure to at least one of the plurality of light wavelengths and wherein the actuation is an exposure of the at least one of the plurality of light wavelengths to the at least one second adhesive portion.

39. The system of claim 38, wherein the exposure includes removing a removable blocking layer from the drape to expose the photosensitive adhesive to ambient light.

40-64. (canceled)
